(19) United States
(12) Patent Application Publication
(10) Pub. No.: US 2021/0106313 A1
(43) Pub. Date: Apr. 15, 2021

(54) METHODS AND SYSTEMS FOR ULTRASOUND IMAGE DISPLAY

(71) Applicant: GE Precision Healthcare LLC, Milwaukee, WI (US)

(72) Inventor: Jungho Kim, Seoul (KR)

(21) Appl. No.: 16/597,596

(22) Filed: Oct. 9, 2019

(51) Int. Cl. A61B 8/08; A61B 8/00; G06T 7/00; G06F 3/048

(52) U.S. Cl. CPC A61B 8/52; A61B 8/463; G06T 2207/10132; G06F 3/048; G06T 7/0012

(57) ABSTRACT

Various methods and systems are provided for generating a set of preset display parameters. In one example, a first set of selectable images are displayed. A selection of a first image from the first set of selectable images may affect display of a second set of selectable images. Selection of images from the rounds may be saved to the set of preset display parameters.

### FIELD

Embodiments of the subject matter disclosed herein relate to display of an ultrasound image.

### BACKGROUND

Ultrasound imaging utilizes high-frequency sound waves to produce images of organs, tissues, or blood flow. The sound waves are produced by an ultrasound probe or transducer and transmitted in pulses. Reflection of the sound waves by boundaries between organs, tissues, bones, etc., are detected by the probe and relayed to a control unit where the reflected waves are converted to a two dimensional image.

An ultrasound imaging system may present an operator with various control settings to allow customizable adjustment of image display settings according to the operator's preferences. The control settings may include parameters such as two-dimensional gain, contrast, and resolution, amongst others. As such, it may be desirable to provide an efficient method to allow the operator to select each display parameter and store the selected settings as a preset for future use.

### BRIEF DESCRIPTION

In one embodiment, a method comprises, during a first round, displaying a first set of selectable images, each image of the first set of selectable images depicting a single scanned image and displayed with a different variation of a first display parameter, receiving a selection of a first image from the first set of selectable images and saving the variation of the first display parameter at which the first image was displayed in a set of preset display parameters. The method further includes, during a second round, displaying a second set of selectable images based on the selection of the first image from the first set, each image of the second set of selectable images depicting the scanned image with a variation of a second display parameter, receiving a selection of a second image from the second set of selectable images and saving the variation of the second display parameter at which the second image was displayed in the set of preset display parameters, and displaying a subsequent image according to the saved set of preset display parameters. In this way, a set of preset display parameters may be generated quickly and efficiently.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows a block diagram of an example of an ultrasound imaging system.

FIG. 2 shows a first set of images showing variations in a first display parameter which may be displayed on a display device of the ultrasound imaging system of FIG. 1 in a sequential selection process.

FIG. 3 shows a second set of images showing variations in a second display parameter.

FIG. 4 shows a third set of images showing variations in a third display parameter.

FIG. 5 shows a block diagram of a single image sequential selection process for generating an image preset.

FIG. 6 shows a first plot depicting dependency of a second display parameter on a previous, first display parameter of a single image sequential selection process.

FIG. 7 shows a second plot depicting dependency of the first display parameter on the second display parameter of the single image sequential selection process.

FIG. 8 shows a third plot depicting an example of a relationship between one display parameter on another that produces discrete and discontinuous ranges of correlation values.

FIG. 9 shows a fourth plot depicting dependency of the first display parameter on a subsequent, third display parameter of the single image sequential selection process.

FIG. 10 shows an example of a method for displaying images obtained with optimized parameters.

FIG. 11 shows an example of a method for a single image sequential selection process that may be executed as part of the method of FIG. 10.

### DETAILED DESCRIPTION

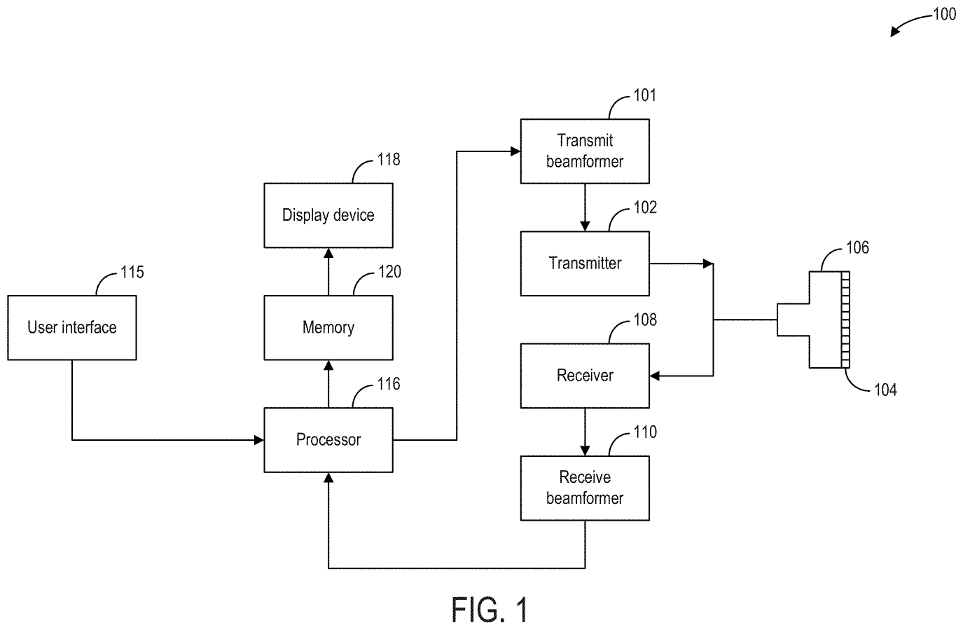

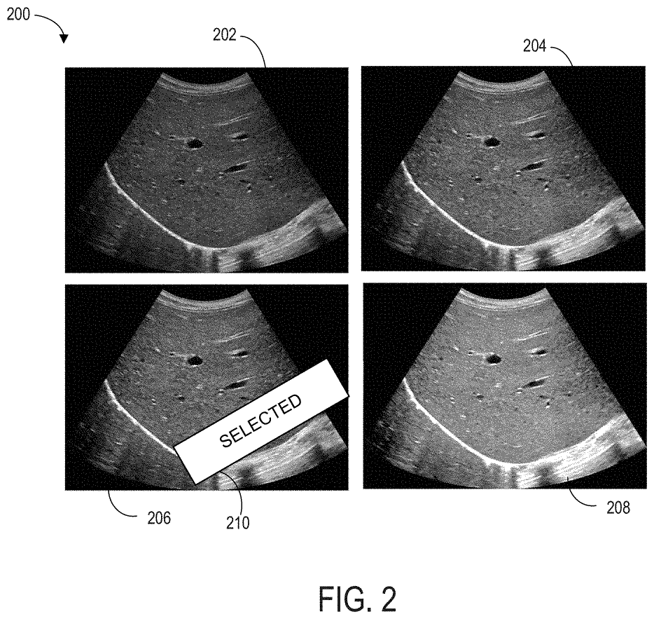

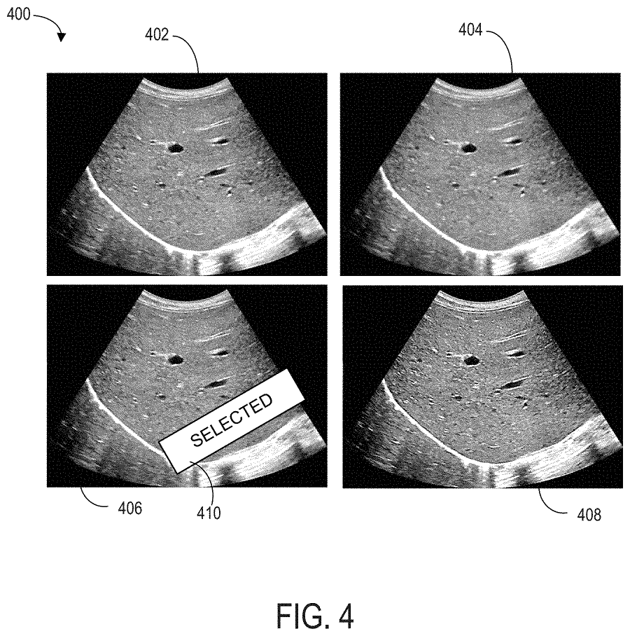

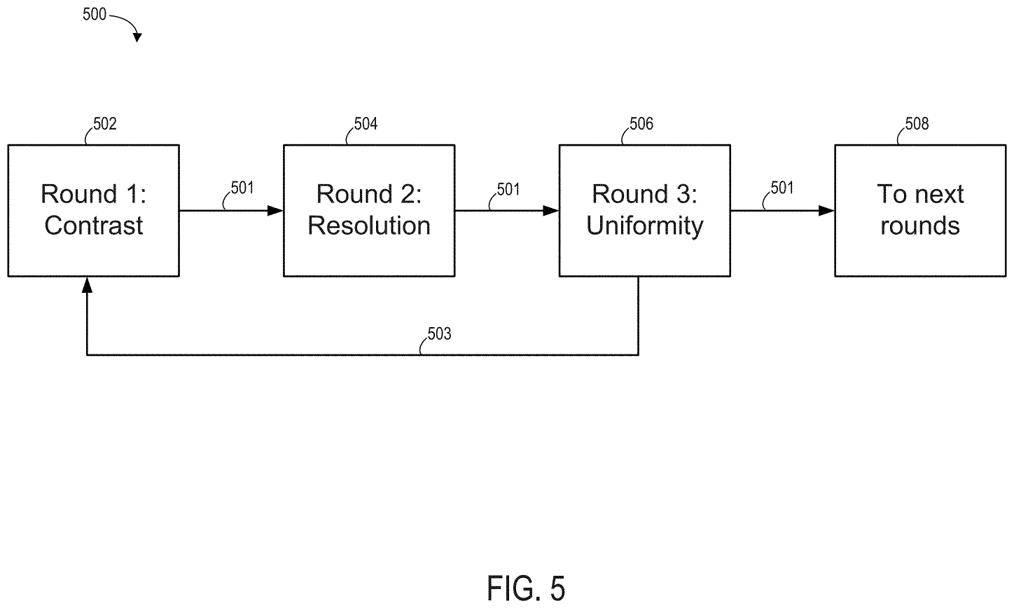

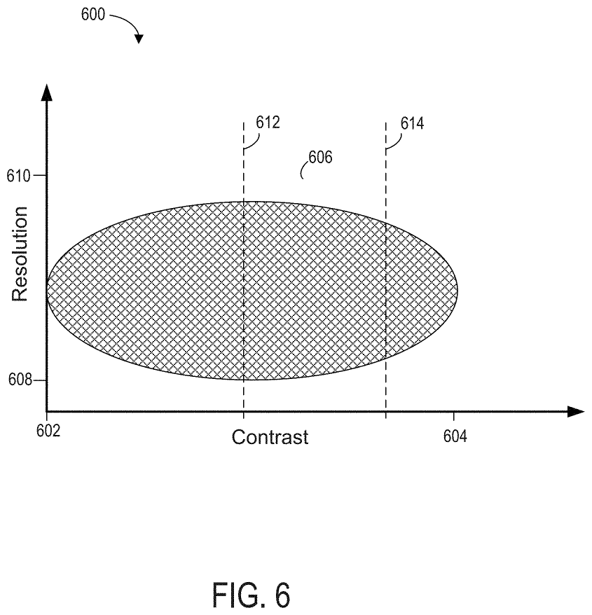

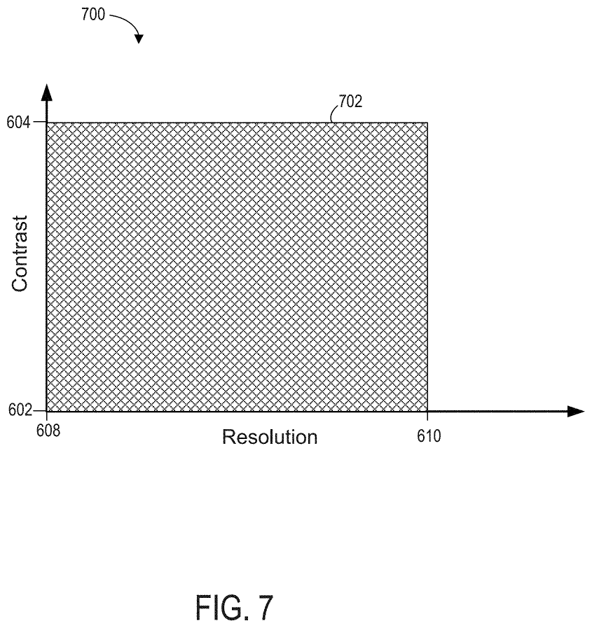

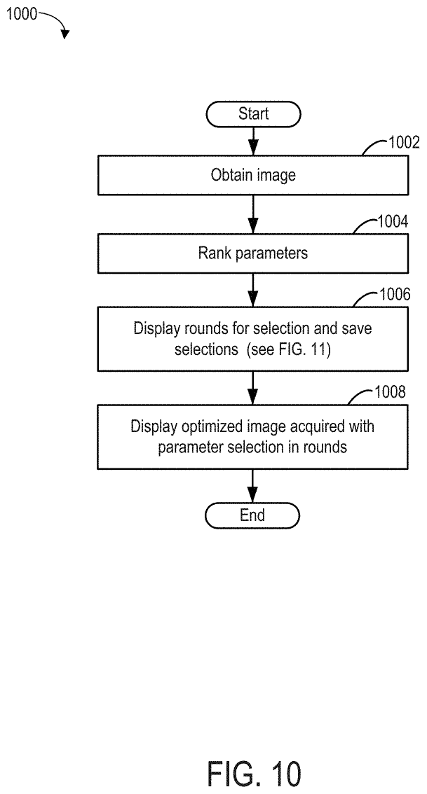

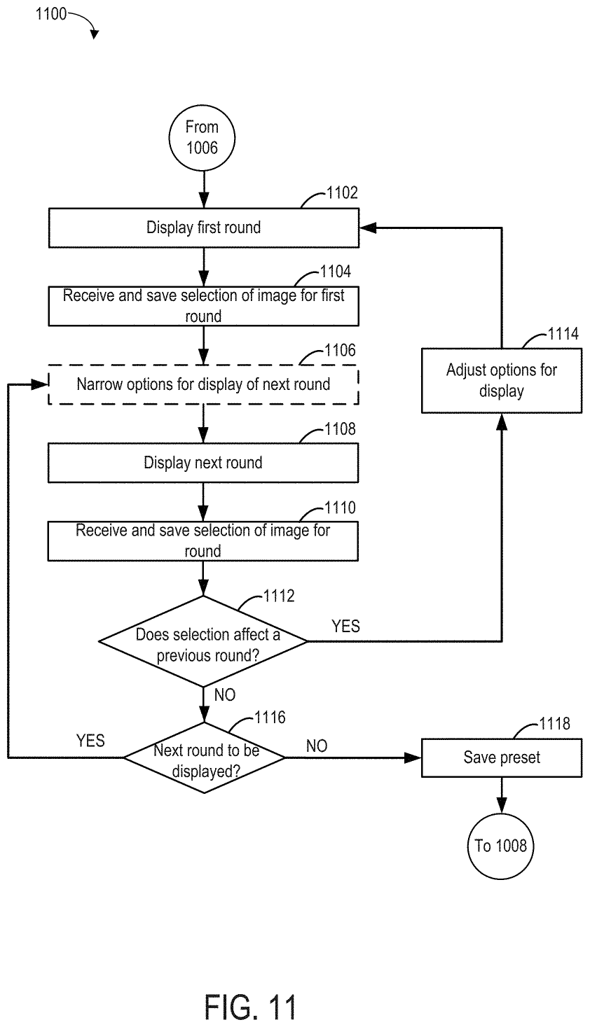

The following description relates to generating presets for displaying ultrasound images. An ultrasound imaging system may utilize ultrasonic signals to obtain images of a patient's tissues and organs. An example of an ultrasound imaging system is shown in FIG. 1. The ultrasound imaging system may include a display device to show a scanned image and display settings may be adjusted via a single image sequential selection process. In the single image sequential selection process, multiple iterations of the image may be displayed simultaneously as a set of images, each iteration depicting a variation in one display parameter category. Each of the image sets may be displayed as a selectable round where an operator may choose an image iteration from a displayed image set. Different image sets, all depicting a common image, are shown in FIGS. 2-4 where each set illustrates adjustments to a specific display parameter. Display settings specific to a particular operator may be saved at a preset through the single image sequential selection process, depicted in FIG. 5 in a block diagram. In some examples, a selection of a first display parameter may affect a selectable range of a second, subsequent display parameter, as illustrated in FIG. 6 in a first graph plotting the second display parameter relative to the first display parameter. FIGS. 7-9 show additional graphs illustrating different relationships between display parameters. The relationships between the display parameters may determine an ordering of display rounds as well as sequential flow of the preset-generating process, as shown in FIGS. 10-11 in an example of a method for the single image sequential selection process.

An ultrasound imaging system may include an ability to adjust a plurality of display settings as commanded by an operator. For example, one operator may prefer coarse-textured images with high contrast while another operator may prefer finer-textured images with lower contrast. Thus the display settings may include numerous adjustable parameters which may be selected by the operator and saved as a preset in a memory of the control unit. The operator may recall the preset during each operation of the ultrasound imaging system and/or adjust the display settings at any time.

A number of parameters which may be adjusted may be large in the ultrasound imaging system. For example, the parameters may include contrast, resolution, two-dimensional gain, dynamic range, frequency, etc. In conventional systems, adjustment of each parameter may be conducted by first obtaining a scanned ultrasound image. The image may be displayed to allow the operator to observe and compare variations in one parameter and choose a desired level or value of the parameter. The process of scanning an image and choosing the parameter value is repeated for each parameter. Depending on a number of display parameters, a period of time spent obtaining an image for selection of each parameter to form the preset may be long and may disrupt a workflow of the operator.

The issues described above may be at least partially addressed by a single image sequential selection process. The process may allow an operator to generate a preset based on a single scanned image which may be used to display iterations of a display parameter. An order of parameter selection may be configured to allow a dependency of, for example, a second parameter on a first parameter to modify a display of the second parameter iterations based on selection of the first parameter. An amount of time spent on generating the preset and/or adjusting the preset may thereby be reduced. Details of the single image sequential selection process are provided further below following a description of an ultrasound imaging system in which the single image sequential selection process may be implemented.

It will be noted that while the single image sequential selection process is described herein with respect to the ultrasound imaging system, the process may be applied to any type of imaging system. For example, the single image sequential selection process may be similarly used to generate a preset for MM, radiography, elastography, tomography, etc.

Referring to FIG. 1, a schematic diagram of an ultrasound imaging system 100 in accordance with an embodiment of the disclosure is shown. The ultrasound imaging system 100 includes a transmit beamformer 101 and a transmitter 102 that drives elements (e.g., transducer elements) 104 within a transducer array, herein referred to as probe 106, to emit pulsed ultrasonic signals (referred to herein as transmit pulses) into a body (not shown). As explained further below, the transducer elements 104 may be comprised of a piezoelectric material. When a voltage is applied to a piezoelectric crystal, the crystal physically expands and contracts, emitting an ultrasonic spherical wave. In this way, transducer elements 104 may convert electronic transmit signals into acoustic transmit beams.

After the elements 104 of the probe 106 emit pulsed ultrasonic signals into a body (of a patient), the pulsed ultrasonic signals are back-scattered from structures within an interior of the body, like blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs ultrasound data. Additionally, transducer element 104 may produce one or more ultrasonic pulses to form one or more transmit beams in accordance with the received echoes.

The terms “scan” or “scanning” may be used in this disclosure to refer to acquiring data through the process of transmitting and receiving ultrasonic signals. The term “data” may be used in this disclosure to refer to either one or more datasets acquired with an ultrasound imaging system. In one embodiment, data acquired via ultrasound system 100 may be used to train a machine learning model. A user interface 115 may be used to control operation of the ultrasound imaging system 100, including to control the input of patient data (e.g., patient medical history), to change a scanning or display parameter, to initiate a probe repolarization sequence, and the like. The user interface 115 may include one or more of the following: a rotary element, a mouse, a keyboard, a trackball, hard keys linked to specific actions, soft keys that may be configured to control different functions, and a graphical user interface displayed on a display device 118.

The ultrasound imaging system 100 also includes a processor 116 to control the transmit beamformer 101, the transmitter 102, the receiver 108, and the receive beamformer 110. The processor 116 is in electronic communication (e.g., communicatively connected) with the probe 106. For purposes of this disclosure, the term “electronic communication” may be defined to include both wired and wireless communications. The processor 116 may control the probe 106 to acquire data according to instructions stored on a memory of the processor, and/or memory 120. The processor 116 controls which of the elements 104 are active and the shape of a beam emitted from the probe 106. The processor 116 is also in electronic communication with the display device 118, and the processor 116 may process the data (e.g., ultrasound data) into images for display on the display device 118. The processor 116 may include a central processor (CPU), according to an embodiment. According to other embodiments, the processor 116 may include other electronic components capable of carrying out processing functions, such as a digital signal processor, a field-programmable gate array (FPGA), or a graphic board.

Some embodiments of the invention may include multiple processors (not shown) to handle the processing tasks that are handled by processor 116 according to the exemplary embodiment described hereinabove. For example, a first processor may be utilized to demodulate and decimate the RF signal while a second processor may be used to further process the data prior to displaying an image. It should be appreciated that other embodiments may use a different arrangement of processors.

The processor 116 may process data according to adjustments commanded by an operator through a single image sequential selection process. The single image sequential selection process may include displaying rounds of images at the display device 118, each image an iteration of a single scanned image. Each round of the images displays a set of iterations varied based on a display parameter to be evaluated. An order of rounds, e.g., an order of displayed parameters, may be based on relationships between the parameters. Upon completion of display parameter selection, a preset may be created which is then stored at a memory 120 of the ultrasound imaging system 100.

The memory 120 is included for storing processed frames of acquired data as well as operator-generated presets. In an exemplary embodiment, the memory 120 is of sufficient capacity to store at least several seconds' worth of frames of ultrasound data. The frames of data are stored in a manner to facilitate retrieval thereof according to its order or time of acquisition. The memory 120 may comprise any known data storage medium.

A single image sequential selection process may be used to generate a preset according to an operator's display preferences through a simple and efficient routine. The process may include first obtaining an ultrasound image through a probe of an ultrasound imaging system, such as the probe 106 of the ultrasound imaging system 100 of FIG. 1. The scanned image, e.g., the original image, may be processed at a processor, e.g., the processor 116 of FIG. 1, and displayed at a display device, e.g., the display device 118 of FIG. 1, as a set of images, where each image of the set of images is an iteration of the original image. Each iteration of the image may depict a variation in a specific display parameter.

For example, a first round of images 200 is shown in FIG. 2, including a first image 202, a second image 204, a third image 206, and a fourth image 208. It will be appreciated that the first round of images 200 is a non-limiting example of how a round of images may be displayed. Other examples may include rounds with any number of images to depict variations in a display parameter. For example, a round of images may include 3, 6, or 10, etc., images without departing from the scope of the present disclosure.

The first round of images 200, as well as each subsequent round of images, refers to display of replicates of the original image where each replicate differs from the other replicates in the round based on a specific display parameter depicted in the round. For example, a liver may be scanned to obtain the original image. The first round of images may display four replicate images of the liver, each replicate image varying, for example, a value of contrast. An operator may select one of the replicate images from the first round. In response to the selection, a second round of images may be immediately displayed, where the second round of images includes four replicate images, each replicate image of the second round showing the original image with the chosen contrast value. In the second round of images, a new display parameter is depicted and varied amongst each image of the second round of images, such that each replicate has a different value of the new display parameter.

As such, each session during which the single image sequential selection process is implemented to generate a preset includes displaying sequential rounds of images, each image showing the liver. The rounds are shown sequentially, immediately following one another in response to selection of an image from a previous round. For example, the second round of images is displayed immediately following display and selection of the first round of images, with no other selections or operations occurring in between, and a third round of images is displayed immediately following display and selection of the second round of images, and so on until all display parameters are shown and selected. The session may then end. A new session may be initiated based on scanning of a new original image, e.g., of a different anatomical region, such as a kidney, or based on an operator request.

Returning to FIG. 2, the first round of images 200 may be displayed at a display device of an ultrasound imaging system and an operator may interact with the first round of images 200 via a user interface, such as the user interface 115 of FIG. 1. Each image of the first round of images 200 may be a replicate of an original image and show an adjustment in a first display parameter according to a level, value, or some other numeric representation of a setting of the first display parameter. The original image may be a scanned image of a liver. The first round of images depict variations in, for example, contrast, where the first image 202 has a contrast value of [50 DB, the second image 204 has a contrast value of 55 dB, the third image 206 has a contrast value of 60 dB, and the fourth image 208 has a contrast value of 65 dB.

An operator may select one of the first round of images 200 by, for example, clicking on one of the images with a pointing device, such as a mouse with a cursor displayed on the display device, or entering a touch input to the display device. As an example, as shown in FIG. 2, the operator may choose the third image 206. Selection of the third image 206 may be indicated by a label 210 and the chosen value of the first display parameter, according to the selected image, is saved to a memory of a control unit of the ultrasound imaging system, where the control unit may include a processor and the memory, such as the processor 116 and the memory 120 of FIG. 1. In other examples, selection of one of the images may be signaled by a change in frame color of the selected image, or highlighting of the selected image, or some other visual indicator.

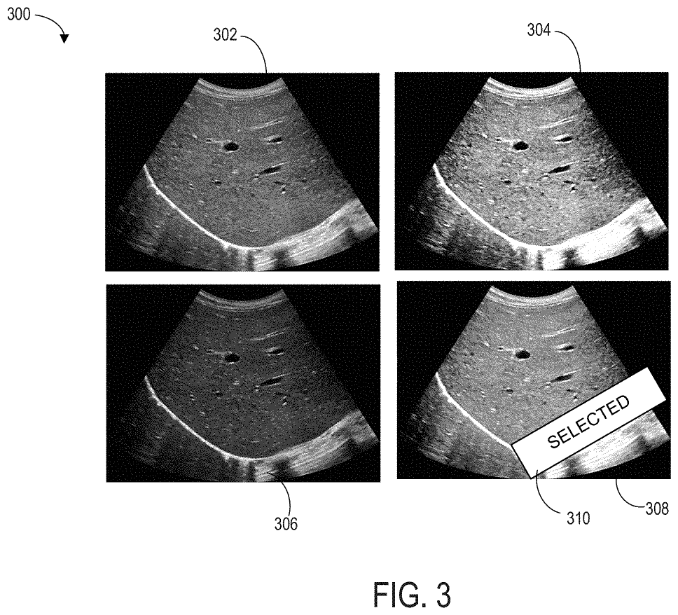

In response to selection of the third image 206 from the first round of images 200 and saving of the selected value of the first display parameter, the single image sequential selection process may immediately display a second round of images 300, as shown in FIG. 3. The second round of images 300 includes a first image 302, a second image 304, a third image 306, and a fourth image 308, each image a replicate of the original image that is also depicted in the first round of images 200 of FIG. 2. A second display parameter is varied in the second round of images 300, each image depicting a value, for example, for a setting of the second display parameter.

The operator may similarly select one image from the second round of images 300. For example, selection of the fourth image 308 results in visual indication 310 of the selection. The chosen display parameter setting from the second round of image 300 may be saved to a same dataset in the memory of the control unit as the selection from the first round of images 200.

A third round of images 400 may then be displayed immediately following selection from the second round of images 300. The third round of images 400 may also include a first image 402, a second image 404, a third image 406, and a fourth image 408, each representing a variation in a value of a third display parameter. Selection of one of the images generates a visual indication 410 of which setting is chosen. The selected parameter value is saved to the dataset.

By using a single scanned image to display each selectable parameter rather than obtaining a new scanned image for evaluation of each parameter, the operator may quickly generate a preset. Each new round of images may be immediately displayed upon selection of a previous round, thereby expediting the selection process. Furthermore, the preset may be easily updated when, for example, updated components of the ultrasound imaging system are installed or if the operator's preferences changes over time.

The single image sequential selection process may continue as described for FIGS. 2-4 until all display parameters are displayed and selected. Selection of the display parameters may, in some examples, be independent of one another, e.g., selection of one parameter does not affect selection of another parameter. In other examples, however, a first parameter may have a relationship with a second parameter where a range of values that may be selectable, e.g., acceptable, of the second parameter is narrowed or focused based on selection of the first parameter.

As such, an order of parameter selection in the single image sequential may be configured to provide a funneling, or at least partially funneling, effect in a downstream direction. For example, a parameter that is not, or is least affected by other display parameters may be selected first. A second round may be based on a parameter that may or may not be affected by selection of the first parameter and less affected by subsequent parameters. A third round may be modified based on one or both of the first and second rounds and less affected by subsequent rounds.

The parameters may therefore be weighted based on a dependency on other parameters and organized to have a focusing, e.g., funneling, effect on subsequent rounds. For example, a block diagram is illustrated in FIG. 5, depicting a flow of a single image sequential selection process as indicated by arrows 501. At 502, a first round may include displaying variations in contrast. The process may continue downstream to 504 after an image is selected from the first round to display a second round of a set of images showing variations in resolution.

A relationship between contrast and resolution is shown in a first graph 600 in FIG. 6. The first graph 600 plots resolution along the y-axis and contrast along the x-axis. A range of contrast values extends between a minimum contrast value 602 to a maximum contrast value 604. Shaded area 606 represents acceptable values of resolution that may be chosen according to values of contrast. The acceptable values of resolution may a target range of values, adjusted based on the contrast value, providing visually desirable variations in resolution, while maintaining a minimum quality of resolution. The acceptable values may further be dependent on a scan plane or view used in obtaining the original scanned image. A range of resolution values extends between a minimum resolution value 608 and a maximum resolution value 610.

A range of acceptable resolution values may be varied based on a selection of a contrast value. For example, the range of acceptable resolution values may encompass a full range of the resolution values, e.g., from the minimum 608 to a value below the maximum 610 when a first contrast value, indicated by dashed line 612, is selected. However, at a second contrast value indicated by dashed line 614, the range of acceptable resolution values may be reduced relative to the range of acceptable resolution values at the first contrast value when the second contrast value is chosen.

Conversely, the resolution value may have little to no effect on an acceptable range of contrast values, as illustrated in a second graph 700 in FIG. 7. The second graph 700 plots contrast along the y-axis and resolution along the x-axis. The minimum contrast value 602 and maximum contrast value 604 is indicated as well as the minimum resolution value 608 and the maximum resolution value 610. A shaded area 702 indicates acceptable contrast values based on the resolution value.

As illustrated by the shaded area 702, the full range of contrast values is acceptable between the minimum value 608 and the maximum value 610 of resolution. Thus, selection of contrast prior to resolution may streamline the single image sequential selection process by narrowing subsequent display parameter ranges when applicable. Ordering of the subsequent display parameters may be determined based on ranking of an effect of one display parameter on one or more other display parameters.

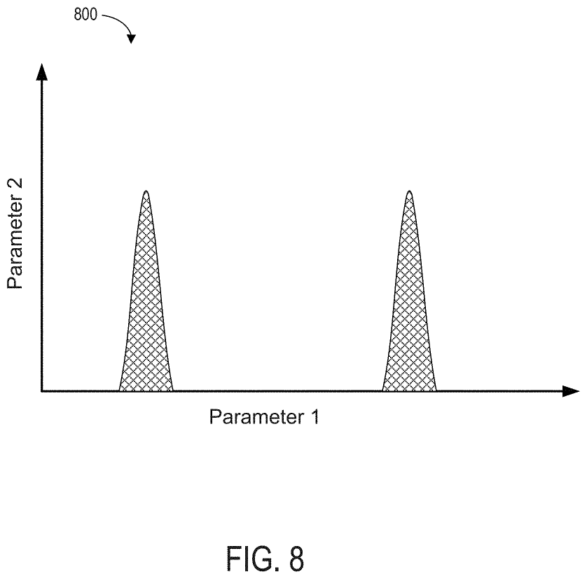

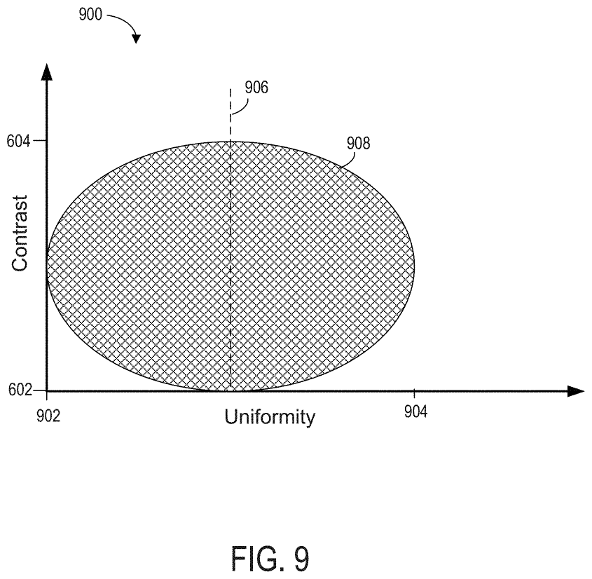

It will be appreciated that the relationship between contrast and resolution depicted in the first graph 600 of FIG. 6 is a non-limiting example of how one display parameter may affect another. In other examples, such as shown in a third graph 800 in FIG. 8, a first parameter, plotted along the x-axis, may allow discrete, discontinuous clusters of a second parameter's values. In yet other examples, various other correlation configurations may be envisioned, e.g., correlation areas forming shapes other than an ellipse as shown in FIGS. 6 and 9.

The selection order of the display parameters may therefore be arranged based on an algorithm implemented by the control unit to rank the parameters based on dependency on other parameters. The selection order may thus begin with a parameter that is least affected by selection of other parameters, providing a broadest range of selectable values, and each sequential parameter may have a narrower range of values than a previous parameter. However, in some examples, a downstream display parameter may have an effect on an upstream display parameter due to interaction of the downstream display parameter with more than one other parameter.

For example, returning to FIG. 5, after selection of an image from the second round at 504, a third round may be displayed at 506, showing variations in uniformity. The uniformity may be narrowed by selection of a resolution value at the second round, with a similar relationship to the relationship between contrast and resolution (e.g., as shown in the first graph 600 of FIG. 6) or may not be affected by selection of the resolution value. However, selection of a uniformity value may have an effect on the contrast acceptable range, as shown in a fourth graph 900 in FIG. 9.

The fourth graph 900 plots contrast along the y-axis and uniformity along the x-axis. The minimum contrast value 602 and the maximum contrast value 604 are shown as well as a minimum uniformity value 902 and a maximum uniformity value 904. At a first uniformity value, indicated by dashed line 906, a full range (e.g., from the minimum value 602 to the maximum value 604) of the contrast values are acceptable, as indicated by shaded area 908. However, if a value of the uniformity (between the minimum value 902 and the maximum value 904) is selected either to the right or to the left of the first uniformity value, a corresponding acceptable range of values is reduced relative to the range at the first uniformity value.

If the selected uniformity value correlates to a narrower range of acceptable contrast values than the full range, the single image sequential selection process, as shown in FIG. 5, may return to the first round at 502, as indicated by arrow 503. The narrower range of acceptable contrast values may be displayed in the set of images shown in the first round, allowing the operator to re-select a desired contrast value. The process continues again to 504 to select the resolution value from the second round of images. The acceptable range of the resolution values may be altered based on a new selected contrast value.

The process continues to the third round and proceeds to display all display parameters of the ultrasound imaging system sequentially. The process may cycle back to a previously selected round at any point during the process if selection of a downstream parameter is deemed to have an effect on an upstream parameter. However, ordering of the rounds to be displayed may be ranked to minimize cycling back to a previous round. In this way, the single image sequential selection process may provide a simple and efficient method for creating a preset. The preset may be generated based on a single scanned image, thus expediting the process in comparison to conventional methods, as discussed above.

An example of a first routine 1000 for obtaining images with optimized parameters is shown in FIG. 10. A second routine 1100, as shown in FIG. 11, is an example of a method for a single image sequential selection process which may be included in the first routine 1000. The first and second routines 1000, 1100 may be implemented as executable instructions in a control unit of an ultrasound imaging system, such as the ultrasound imaging system of FIG. 1, where the control unit may include a processor and memory such as the processor 116 and memory 120 of FIG. 1. As one example, the first and second routines 1000, 1100 may be implemented in non-transitory memory of the control unit of the ultrasound imaging system.

Turning now to FIG. 10, the first routine 1000 includes obtaining an image at 1002. The image may be obtained by scanning a patient with an ultrasound probe and processing ultrasound signals into an image at the processor of the control unit. The single image may be used to depict one or more display parameters and displayed in rounds, each round showing variations in one parameter.

At 1004, the first routine 1000 includes ranking the parameters to determine an order of display. In one example, ranking the parameters is initiated in response to obtaining the scanned image, indicating a start to new scanning session. In another example, ranking of the parameters may commence in response to a user request to adjust a saved preset or to create a new preset. The ranking may be based on interdependency of a parameter on other parameters and how selection of a parameter value may affect a range of possible values for another parameter. For example, the control unit may be implemented with look-up tables providing correlations between the parameters. The parameters may then be ranked from least affected by other parameters to most affected and displayed according to that order.

In some examples, the ordering of the parameters may be affected by which anatomical region is scanned or based on a diagnostic goal of an examination. For example, parameter ordering of lesion detection versus lesion growth, or an echocardiogram versus a fetal ultrasound may be result in different ranking of parameters.

The first routine 1000 continues to 1006 to display the rounds for selection by an operator. Each selection may be saved into the memory of the control unit and assigned to a dataset for a specific preset. The first routine 1006 proceeds to execute the display of rounds based on the single image according to the second routine 1100, as depicted in FIG. 11.

At 1102, the second routine 1100 includes displaying a first round based on a first parameter determined to be least affected by selection of other parameters. If an already saved preset is used, a range of acceptable values of each display parameter may be set based on selections made from the saved preset and further adjusted as desired when the saved preset is recalled. If no saved preset is recalled, a new preset may be generated via the second routine 1100 and the display parameters may set to default acceptable ranges of values. The default acceptable ranges of values may be a broadest range of values that may be acceptable prior to narrowing by parameter adjustment.

Displaying the first round at 1102 includes showing a set of images, each image using the single scanned image and showing a different value of the first parameter. The values of the first parameter shown may be determined by, for example, a simple calculation dividing the range of acceptable values by a number of selectable images shown. As such, uniform increments of change in the acceptable values are displayed amongst the set of images. An operator may choose an image from the set of images of the first round, e.g., by clicking on the image with an input device. The selection is received and saved into the memory of the control unit at 1104.

The control unit evaluates whether the selection of the first round affects display of a next round, where variations of the image are displayed based on a different display parameter. For example, the first parameter may be contrast and the next parameter may be resolution. The control unit may refer to a look-up table such as the first graph 600 of FIG. 6 and determine what range of values for resolution are acceptable based on the selection of contrast.

The second routine 1100 may proceed to 1106 to adjust or narrow the range of values of the next round for display according to a relationship between the first parameter and the next parameter. However, if the next round is determined to have no effect on the first round, the second routine 1100 may continue directly to 1108 to display the next round. The operator's selection from the most recently displayed round is received and saved to the dataset at 1110. Furthermore, the selection from the most recently displayed round is used to display the next parameter. For example, if a contrast value of 50% is chosen in the first round, the images displayed in the next round all show the contrast value of 50%. As such, the images in the next round shown variations in another parameter, such as resolution, based on the contrast value of 50%. Subsequent rounds may similarly incorporate all previously selected parameters values as a base image to display variations in a new parameter.

At 1112, the second routine 1100 includes determining if the selection from the most recently selected round has an effect on a previous round. If the most recently selected round is determined to affect a previous round, the acceptable range of values are adjusted for the affected previous round at 1114 and the second routine 1100 returns to 1102 to display the previous round with a new set of images, the new set of images also based on the single scanned image.

It will be noted that while 1102 describes display of the first round, the first round may refer to any previous round and the second routine 1100 may return to any previous round at 1112 as the second routine 1100 continues displaying sequential sets of rounds. For example, when a fourth round is displayed, the second routine 1100 may return to any of the first, second, or third rounds, depending on which round is affected by selection of an image from the fourth round.

If the selection from the most recently selected round does not affect a previous round at 1112, the second routine 1100 continues to 1116 to determine if a subsequent round is to be displayed. If another round depicting another parameter is to be displayed, the second routine 1100 returns to 1106 to optionally narrow or adjust an acceptable range of values for the next round or continue directly to 1108 if the next round is not affected by selection of the previous round.

If no next round is to be displayed, the second routine proceeds to 1118 to save the all the selections for each parameter to a dataset to generate the preset. The second routine 1118 then continues to 1008 of the first routine 1000. At 1008, the first routine 1000 includes displaying an optimized image based on the preset incorporating all the selections from the rounds. Further, subsequent ultrasound images acquired via the ultrasound probe may be displayed using the preset generated according to the first routine 1000. The first routine 1000 then ends.

In this way, a preset may be efficiently created via a single image sequential selection process according to an operator's image display preferences. Generation of the preset may include performing a scan with an ultrasound probe to acquire a single image (referred to as the scanned image) and using the scanned image to provide rounds of images, each round associated with a specific display parameter. A set of images, each image a replicate of the scanned image, is displayed in each round to show a range of acceptable values of the display parameter. The rounds may be ordered according to a dependency of a parameter on other parameters, with a parameter least affected by the other parameters assigned as a first round. Subsequent rounds may be increasingly codependent on other parameters, at least in some examples. The single image sequential selection process allows the process to return to previous rounds if a downstream selection is determined to affect an acceptable range of a previously selected parameter. Thus the preset may be quickly and easily generated without demanding scanning of multiple images. In some examples, once a preset has been set according to the sequential selection process described herein, additional adjustments may be made to the preset in response to operator request. For example, the operator may request to update the preset and a new sequential selection process may be initiated. In other examples, the operator may adjust the preset (set according to the sequential selection process) using a graphical user interface (e.g., via selection of user interface control buttons or menus on the graphical user interface) or via input buttons on the ultrasound probe or other location of the ultrasound system.

The technical effect of implementing the single image sequential selection process in a medical imaging system is that an operator preset may be created efficiently, thereby increasing a workflow efficiency and reducing an amount of time spent obtaining scanned images to customize the preset. Furthermore, the preset may be easily updated when operating conditions or instrument conditions are modified.

An embodiment for a method includes, during a first round, displaying a first set of selectable images, each image of the first set of selectable images depicting a single scanned image and displayed with a different variation of a first display parameter and receiving a selection of a first image from the first set of selectable images and saving the variation of the first display parameter at which the first image was displayed in a set of preset display parameters; during a second round, displaying a second set of selectable images based on the selection of the first image from the first set, each image of the second set of selectable images depicting the scanned image and displayed with a different variation of a second display parameter, and receiving a selection of a second image from the second set of selectable images and saving the variation of the second display parameter at which the second image was displayed in the set of preset display parameters; and displaying a subsequent image according to the saved set of preset display parameters.

In a first example of the method, selecting the image from the first set adjusts an acceptable range of values of the second display parameter and wherein the different variations of the second display parameter at which the second set of selectable images are displayed are selected from the acceptable range of values of the second display parameter. In a second example of the method, which optionally includes the first example, selecting the image from the first set narrows the acceptable range of values of the second display parameter. In a third example of the method, which optionally includes one or both of the first and second examples, the method further comprises, after an image is selected from the second set of selectable images, sequentially displaying one or more additional sets of images, each set of images an additional round for display, each of the one or more additional sets of images depicting the scanned image and displayed with a different variation of a different display parameter. In a fourth example of the method, which optionally includes one or more or each of the first through third examples, displaying the one or more additional sets of images includes generating sets of images, each image a replicate of the single scanned image and each of the sets of images depicting a variation within an acceptable range of values in the different display parameter. In a fifth example of the method, which optionally includes one or more or each of the first through fourth examples, the method further includes narrowing the range of acceptable values of each of the additional sets of images based on selection of an image from a previous set of selectable images. In a sixth example of the method, which optionally includes one or more or each of the first through fifth examples, narrowing the range of acceptable values of each of the additional sets of selectable images includes referring to a predetermined relationship between a third display parameter displayed in one of the additional sets of selectable images and a fourth display parameter displayed in the previous set of selectable images. In a seventh example of the method, which optionally includes one or more or each of the first through sixth examples, the method further includes returning to a previously selected set of selectable images when selection of an image of a subsequent set of selectable images of the additional sets of images alters an acceptable range of values of a display parameter of the previously selected set of selectable images. In an eighth example of the method, which optionally includes one or more or each of the first through seventh examples, generating the preset includes receiving and saving the selections from the additional sets of selectable images in addition to the selections of the first and second sets of selectable images and displaying the single scanned image incorporating each of the selections from each of the first, second, and additional set of selectable images. In a ninth example of the method, which optionally includes one or more or each of the first through eighth examples, displaying the second set of selectable images based on the selection of the first image from the first set comprises displaying each image of the second set of selectable images at the variation of the first display parameter at which the first image was displayed.

An embodiment is directed to a method for displaying ultrasound images, the method including scanning a subject to obtain a first image; ranking a plurality of display parameters, each display parameter configured to adjust a display of the first image; sequentially displaying sets of images for selection in rounds, each set of images including replicates of the first image, each replicate of a respective set of images displayed at a different variation of a respective display parameter, wherein an ordering of the sequentially displayed sets of images is based on the ranking of the plurality of display parameters; and presenting a second image based on a respective selection from each round. In a first example of the method, ranking the plurality of display parameters includes referring to effects of selection of one of the plurality of display parameters on an acceptable range of values on another of the plurality of display parameters and ordering the plurality of display parameters from a display parameter least affected by other display parameters to a display parameter most affected by other display parameters. In a second example of the method, which optionally includes the first example, each replicate of the respective set of images is displayed at a different acceptable value of the respective display parameter. In a third example of the method, which optionally includes one or both of the first and second examples, for a first round, the different acceptable values are included in that round based on a selection of an image from a prior round. In a fourth example of the method, which optionally includes one or more or each of the first through third examples, presenting the second image includes displaying the first image at a selected different value for each respective display parameter based on the respective selection from each round. In a fifth example of the method, which optionally includes one or more or each of the first through fourth examples, the method further includes saving, to a memory of a control unit, a preset according to the respective selection from each round for application to subsequent displayed images. In a sixth example of the method, which optionally includes one or more or each of the first through fifth examples, the scanning the subject to obtain the first image includes scanning the subject with an ultrasound probe.

An embodiment for an imaging system is provided. The imaging system includes a device configured to obtain a first image and a control unit with a memory storing executable instructions that, when executed, cause the control unit to: sequentially display a plurality of rounds of images, each round of images including the same image displayed at two or more different values for a respective display parameter of a set of display parameters; assemble a set of preset display parameters that includes a selected value for each display parameter of the set of display parameters based on selection of an image from each round of images, where for at least one round of images, the two or more different values for that respective display parameter are selected based on a selection of an image from a prior round of images; and display a subsequent image according to the set of preset display parameters. In a first example of the imaging system, the imaging system is an ultrasound imaging system, and the device configured to obtain the first image is an ultrasound probe. In a second example of the imaging system, which optionally includes the first example, the set of preset display parameters is stored at the memory of the control unit.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
during a first round,
displaying a first set of selectable images, each image of the first set of selectable images depicting a single scanned image and displayed with a different variation of a first display parameter;
receiving a selection of a first image from the first set of selectable images and saving the variation of the first display parameter at which the first image was displayed in a set of preset display parameters;
during a second round,
displaying a second set of selectable images based on the selection of the first image from the first set, each image of the second set of selectable images depicting the scanned image and displayed with a different variation of a second display parameter;
receiving a selection of a second image from the second set of selectable images and saving the variation of the second display parameter at which the second image was displayed in the set of preset display parameters; and
displaying a subsequent image according to the saved set of preset display parameters.

2. The method of claim 1, wherein selecting the image from the first set adjusts an acceptable range of values of the second display parameter and wherein the different variations of the second display parameter at which the second set of selectable images are displayed are selected from the acceptable range of values of the second display parameter.

3. The method of claim 2, wherein selecting the image from the first set narrows the acceptable range of values of the second display parameter.

4. The method of claim 1, further comprising after an image is selected from the second set of selectable images, sequentially displaying one or more additional sets of images, each set of images an additional round for display, each of the one or more additional sets of images depicting the scanned image and displayed with a different variation of a different display parameter.

5. The method of claim 4, wherein displaying the one or more additional sets of images includes generating sets of images, each image a replicate of the single scanned image and each of the sets of images depicting a variation within an acceptable range of values in the different display parameter.

6. The method of claim 5, further comprising narrowing the range of acceptable values of each of the additional sets of images based on selection of an image from a previous set of selectable images.

7. The method of claim 6, wherein narrowing the range of acceptable values of each of the additional sets of selectable images includes referring to a predetermined relationship between a third display parameter displayed in one of the additional sets of selectable images and a fourth display parameter displayed in the previous set of selectable images.

8. The method of claim 7, further comprising returning to a previously selected set of selectable images when selection of an image of a subsequent set of selectable images of the additional sets of images alters an acceptable range of values of a display parameter of the previously selected set of selectable images.

9. The method of claim 8, wherein generating the preset includes receiving and saving the selections from the additional sets of selectable images in addition to the selections of the first and second sets of selectable images and displaying the single scanned image incorporating each of the selections from each of the first, second, and additional set of selectable images.

10. The method of claim 1, wherein displaying the second set of selectable images based on the selection of the first image from the first set comprises displaying each image of the second set of selectable images at the variation of the first display parameter at which the first image was displayed.

11. A method for displaying ultrasound images, comprising:
scanning a subject to obtain a first image;
ranking a plurality of display parameters, each display parameter configured to adjust a display of the first image;
sequentially displaying sets of images for selection in rounds, each set of images including replicates of the first image, each replicate of a respective set of images displayed at a different variation of a respective display parameter, wherein an ordering of the sequentially displayed sets of images is based on the ranking of the plurality of display parameters; and
presenting a second image based on a respective selection from each round.

12. The method of claim 11, wherein ranking the plurality of display parameters includes referring to effects of selection of one of the plurality of display parameters on an acceptable range of values on another of the plurality of display parameters and ordering the plurality of display parameters from a display parameter least affected by other display parameters to a display parameter most affected by other display parameters.

13. The method of claim 11, wherein each replicate of the respective set of images is displayed at a different acceptable value of the respective display parameter.

14. The method of claim 13, wherein, for a first round, the different acceptable values are included in that round based on a selection of an image from a prior round.

15. The method of claim 11, wherein presenting the second image includes displaying the first image at a selected different value for each respective display parameter based on the respective selection from each round.

16. The method of claim 11, further comprising saving, to a memory of a control unit, a preset according to the respective selection from each round for application to subsequent displayed images.

17. The method of claim 11, wherein the scanning the subject to obtain the first image includes scanning the subject with an ultrasound probe.

18. An imaging system, comprising:
a device configured to obtain a first image;
a control unit with a memory storing executable instructions that, when executed, cause the control unit to:
sequentially display a plurality of rounds of images, each round of images including the same image displayed at two or more different values for a respective display parameter of a set of display parameters;
assemble a set of preset display parameters that includes a selected value for each display parameter of the set of display parameters based on selection of an image from each round of images, where for at least one round of images, the two or more different values for that respective display parameter are selected based on a selection of an image from a prior round of images; and
display a subsequent image according to the set of preset display parameters.

19. The imaging system of claim 18, wherein the imaging system is an ultrasound imaging system.

20. The imaging system of claim 18, wherein the set of preset display parameters is stored at the memory of the control unit.
